(19) United States
(12) Patent Application Publication
(10) Pub. No.: US 2003/0225526 A1
(43) Pub. Date: Dec. 4, 2003

(54) Molecular cancer diagnosis using tumor gene expression signature

(76) Inventors: Todd R. Golub, Newton, MA (US); Sayan Mukherjee, Cambridge, MA (US); Sridhar Ramaswamy, Brookline, MA (US); Ryan Rifkin, Cambridge, MA (US); Pablo Tamayo, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/294,453

(22) Filed: Nov. 14, 2002

Related U.S. Application Data
(60) Provisional application No. 60/332,268, filed on Nov. 14, 2001.

(51) Int. Cl. G06F 19/00; G01N 33/48; G01N 33/50

(52) U.S. Cl. 702/19

(57) ABSTRACT

Methods are provided for the clssification of disease types (e.g., cancer types), outcome predictions, and treatment classes based on algorithmic classifiers used to analyze large datasets.

### RELATED APPLICATION

This application claims the benefit of U.S. Provisional Application No. 60/332,268, filed Nov. 14, 2001.

The entire teachings of the above application are incorporated herein by reference.

### GOVERNMENT SUPPORT

The invention was supported, in whole or in part, by training grant 5T32 HL07623 from the National Institutes of Health. The Government has certain rights in the invention.

### BACKGROUND OF THE INVENTION

The accurate classification of human cancer based on anatomic site of origin is an important component of modern cancer treatment. It is estimated that more than 40,000 cancer cases per year in the U. S. are difficult to classify using standard clinical and histopathologic approaches. Molecular approaches to cancer classification have the potential to effectively address these difficulties. However, decades of research in molecular oncology have yielded few useful tumor-specific molecular markers. An important goal in cancer research, therefore, continues to be the identification of tumor specific genetic markers and the use of these markers for molecular cancer classification.

Using traditional methods, such as morphology analyses, histochemical analyses, immunophenotyping and cytogenetic analyses, often only one or two characteristics of the sample are analyzed to determine the sample's classification, resulting in inconsistent and sometimes inaccurate results. Such results can lead to incorrect diagnoses and potentially ineffective or harmful treatment. For example, optimal treatment of cancer patients depends on establishing accurate clinico-pathologic diagnoses. In some instances, this has proven difficult or impossible due to atypical clinical presentations or uncharacteristic histopathology. A large part of cancer classification relies upon clinical judgment and microscopic tissue examination with an eye toward placing tumors in currently accepted categories based on a tumor's tissue of origin. This approach is subjective and therefore variable even among experienced clinicians and pathologists. In addition, there is a wide spectrum of cancer morphology and many tumors are atypical or lack morphologic features that are useful for differential diagnosis. These difficulties result in estimated diagnostic error rates of 2% to 8% (25,000-100,000 cases per year in the United States), and have prompted calls for mandatory second opinions in all surgical pathology cases.

Oligonucleotide microarray-based gene expression profiling allows investigators to study the simultaneous expression of thousands of genes in biological systems. In principle, tumor gene expression profiles can serve as molecular fingerprints that allow for the accurate and objective classification of tumors. The classification of primary solid tumors is a difficult problem due to limitations in sample availability, identification, acquisition, integrity, and preparation. Moreover, a solid tumor is a heterogeneous cellular mix, and gene expression profiles might reflect contributions from non-malignant components, further confounding classification. In addition, there are intrinsic computational complexities in making multi-class, as opposed to binary class, distinctions. Thus, a need exists for accurate markers and methods for identifying tumor classes and classifying tumor samples. At present, comprehensive gene expression databases have yet to be developed, and there are no established analytical methods capable of solving complex, multi-class, gene expression-based classification problems.

### SUMMARY OF THE INVENTION

The present invention is directed, in part, to methods for classifying biological samples, including, for example, tumor samples.

In one embodiment, the invention is directed to a method of classifying a biological sample comprising: determining the expression pattern of one or more markers in a sample; providing a model generated by a supervised learning algorithm based on a dataset of expression values from known biological classes; and comparing the expression pattern of the markers in the sample to the model, thereby classifying said biological sample. In one embodiment, the biological sample can be classified either as a disease sample or normal sample. In a preferred embodiment, the dataset contains expression values from multiple known biological classes. In one embodiment, the disease state can be cancer, coronary artery disease, neurodegenerative disease or pulmonary disease. In one embodiment, the dataset includes data from known classes of a particular disease. In an embodiment where the disease is cancer, the classes of cancer can include, for example, breast adenocarcinoma, prostate adenocarcinoma, lung adenocarcinoma, colorectal adenocarcinoma, lymphoma, bladder transitional cell carcinoma, melanoma, uterine adenocarcinoma, leukemia, renal cell carcinoma, pancreatic adenocarcinoma, ovarian carcinoma, pleural mesothelioma and central nervous system. In a particular embodiment, a digital processor is used to compare the expression pattern of the markers in the sample to the model.

In one embodiment, the biologic sample is compared to the model in a pairwise manner, e.g., a one versus all other comparison, for each biological class. In one embodiment, the supervised learning algorithm can be a support vector machine algorithm. The support vector machine algorithm can be, for example, either linear or non-linear. The steps of the methods described herein can be performed in a computer system.

In another embodiment, the invention is directed to, in a computer system, a method for classifying at least one sample to be tested that is obtained from an individual, wherein expression values of more than one marker are determined for the sample to be tested, comprising: receiving the gene expression values for more than one marker in the sample to be tested; means for providing a model generated by a supervised learning algorithm based on a dataset of expression values from known biological classes; comparing the gene expression values of the sample to that of the model, to thereby produce a classification of the sample; and providing an output indication of the classification.

In another embodiment, the invention is directed to a computer apparatus for providing an indication of the classification of a biological sample, wherein the sample is obtained from an individual, wherein the apparatus includes: a source of expression values of more than one marker in the sample; means for providing a model generated by a trained algorithm based on a dataset of expression values from known biological classes; a processor routine executed by a digital processor, coupled to receive the expression values from the source, the processor routine determining classification of the sample by comparing the expression values of the sample to the model; and an output assembly, coupled to the digital processor, for providing an indication of the classification of the sample.

In another embodiment, the invention is directed to a method of determining a treatment plan for an individual having a disease, including: obtaining a sample from the individual; providing a model generated by a supervised learning algorithm based on a dataset of expression values from known biological classes; assessing the sample for the level of expression of more than one marker; using the model to perform one or more pairwise comparisons of the sample versus at least one disease class, thereby resulting in the classification of the sample; and using the disease class to determine a treatment plan.

In another embodiment, the invention is directed to a method of determining the efficacy of a drug for disease treatment, including: obtaining a sample from an individual having the disease; subjecting the sample to the drug; assessing the drug-exposed sample for the level of expression of more than one marker; providing a model generated by a supervised learning algorithm based on a dataset of expression values from known samples on which the drug has different levels of efficacy; and using a computer to compare the drug-exposed sample to the model to determine the efficacy of the drug in treating the disease. In another embodiment, samples can be obtained at different time points before and after treatment, such that, upon comparison to the model, treatment efficacy can be monitored.

In another embodiment, the invention is directed to a model based on a dataset of expression data comprising a plurality of markers from known biological samples formed using a trained algorithm to define a hyperplane that characterizes a biological class.

In yet another embodiment, the invention is directed to a method of classifying a biological sample including the steps of: determining the expression pattern of one or more markers in a sample; providing a model generated by a linear support vector machine algorithm based on a dataset of expression values from known biological classes; and using a digital processor to compare the expression pattern of the markers in the sample to the model using one or more one versus all other pairwise comparisons, thereby classifying said biological sample.

### BRIEF DESCRIPTION OF THE DRAWINGS

FIG. 1 is a schematic representation of a typical experimental protocol.

FIG. 2 is a schematic representation of the steps involved in multi-class classification.

FIG. 3 is a graphical representation showing the mean classification accuracy and standard deviation plotted as a function of number of genes used by the classifier. The prediction accuracy decreases with a decreasing number of genes.

FIG. 4 is a diagram depicting hierarchical clustering: 144 tumors spanning 14 tumor classes were clustered according to their gene expression patterns. BR breast adenocarcinoma, PR prostate adenocarcinoma, LU lung adenocarcinoma, CO colorectal adenocarcinoma, LY lymphoma, BL bladder transitional cell carcinoma, ML melanoma, UT uterine adenocarcinoma, LE leukemia, RE renal cell carcinoma, PA pancreatic adenocarcinoma, OV ovarian carcinoma, ME pleural mesothelioma, CNS central nervous system.

FIG. 5 is a schematic showing a general classification strategy. The multi-class cancer classification problem is divided into a series of 14 one class versus all other classes (OVA) problems, where each OVA problem is addressed by a different class-specific classifier (e.g., “breast cancer” versus “all other”). Each classifier uses the support vector machine (SVM) algorithm to define a hyperplane that best separates training samples in these two classes. Test samples are sequentially presented to each of 14 OVA classifiers and the sample's class is determined by the classifier with the highest confidence, as determined by the distance from the hyperplane. In the example shown, the sample is predicted to be breast cancer.

FIGS. 6A-C are graphical representations of data used in the classification of tumor samples. FIG. 6A is a scatter plot showing SVM OVA classifier confidence as a function of correct calls (left) or errors (right) for Training and Test samples. FIG. 6B is a histogram showing classification confidence and accuracy. FIG. 6C shows the accuracy as a function of first, second, and third highest OVA classifier predictions.

FIG. 7 depicts quantitative displays of accuracy results for the OVA/SVM classifier. Top: a table showing results of Training and two test samples (Independent Test Set and Poorly-Differentiated adenocarcinomas (PD)). Bottom: a scatter plot showing SVM OVA classifier confidence as a function of correct calls (left) or errors (right) for the Training and two test samples.

FIGS. 8A and 8B are graphical representations of confusion matrices for the OVA/SVM classifier based on the samples described in FIG. 7. The confusion matrices for the “Train” and “Test” sets are shown.

### DETAILED DESCRIPTION OF THE INVENTION

A description of preferred embodiments of the invention follows.

Disease classification and diagnoses can be difficult, particularly when the molecular mechanisms that lead to the disease are complicated. Cancer is a disease with a very complex set of molecular determinants, and, therefore, poses particular diagnostic and treatment challenges for physicians. Because of its complex molecular nature, accurate classification based on the gene expression of one or a limited number of “informative genes”, used herein to refer to genes that are used to detect or predict a certain phenotype, is often ineffective.

Cancer or disease classification involving many classes, tissue types and informative genes, exhibits increased dimensionality with respect to datasets, thus making multi-class classifications challenging. Difficulties attributed to the small but significant uncertainty in the original labelings, the noise in the experimental and measurement processes, the intrinsic biological variation from specimen to specimen, and the small number of examples, have led to inaccurate diagnoses. The methods described herein, however, allow for remarkably accurate predictions.

The present invention is directed to methods for “molecular diagnostics,” used herein to refer to the process of determining biological classes based on expression patterns of particular markers in biological samples. As used herein, “markers” refer to DNA sequences that allow for the production of mRNA. Such markers can be detected quantitatively and efficiently using “microarrays” (used herein to refer to solid substrates with oligonucleotides complementary to marker mRNA physically attached to the substrate at particular positions). The use of expression data to classify biological samples allows for the accurate determination of class even in cases where one biological class is very similar morphologically to another biological class. The methods described herein rely on models constructed using, e.g., a supervised learning algorithm as a way of analyzing large datasets of expression values of several markers. In particular, this approach can be used to classify a sample as derived from a phenotypic source such as a disease class (e.g., cancer, coronary artery disease, neurodegenerative disease and pulmonary disease) as distinguished from another phenotypic source (e.g., another disease class or normal tissue).

Molecular diagnostics have had only a limited impact on cancer diagnosis because characteristic molecular markers for most solid tumors have yet to be identified (Connolly, J. et al., 1997. in Principles of Cancer Pathology, Holland, J. et al., eds. Williams and Wilkins, Baltimore, Md. 533-555). This has precluded a systematic approach to the molecular classification of human cancers. DNA microarrays have been utilized as a means of collecting expression data as part of a potential strategy for cancer diagnosis based on expression profiles. However, these studies have been limited to a few cancer types and have spanned multiple technology platforms, complicating comparison among different datasets (Golub, T. et al., 1999. Science. 286:531-537; Alizadeh, A. et al., 2000. Nature. 403:503-511; Bittner, M. et al., 2000. Nature. 406:536-540; Perou, C. et al., 2000. Nature. 406:747-752; Hedenfalk, I. et al., 2001. N. Engl. J Med. 344:539-548; Khan, J. et al., 2001. Nat. Med. 7:673-679).

Methods for determining normal versus cancer tissue and methods of cancer diagnosis across all of the common malignancies based on a single reference database is described herein. Databases containing expression profiles from multiple markers can contain expression data from different sets of markers and/or from different pre-determined biological samples (e.g., tumors, coronary artery disease samples, neurodegenerative disease samples, and pulmonary disease samples). Thus, databases can contain expression data that is suited to the particular classification of interest (e.g., classification of cancer types, disease types, or any classifiable phenotype).

The method of the present invention is related in part to analyzing data in large datasets. The datasets used in the present invention contain expression data from a large number of markers expressed in different tissue samples. Expression data can be obtained by a variety of methods known in the art. For example, expression data can be obtained by determining the level of polypeptide products from a particular marker or by quantitatively determining the level of any expression product such as, for example, RNA. The dataset itself is the accumulation of all or any subset of such expression data as collected by any method known in the art.

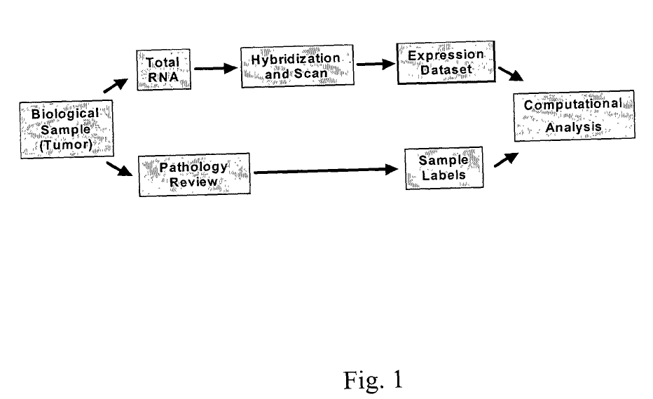

In one embodiment (see FIG. 1), RNA from whole tumors can be used to prepare “hybridization targets” according to published methods (Golub, T. et al., 1999. Science. 286:531-537). Expression profiles for multiple markers, or “target” RNA molecules, can be obtained by detecting the cellular level of RNA corresponding to each marker. This can be performed by isolating RNA from specific cell or tissue types, and quantitatively detecting specific RNA molecules by hybridization to complementary oligonucleotides. For example, hybridization assays using microarrays containing oligonucleotides complementary to specific marker mRNA transcripts arranged on gene chips available from Affymetrix, Inc. (Santa Clara, Calif.) can be used to quantitatively detect RNA levels corresponding to thousands of markers in a single assay. Expression data can be obtained by assaying for the level of a gene expression product (e.g., RNA, peptide or protein). For example, a large expression database containing the expression profiles of more than 16,000 markers from 218 tumor samples representing 14 common human cancer classes was created as a suitable database for use in methods described herein.

Targets can be hybridized sequentially to oligonucleotide microarrays containing, in one embodiment, probe sets representing known DNA sequences. Typical microarrays include, for example, Affymetrix Hu6800 and Hu35KsubA GeneChips™. For these chips, arrays are scanned using commercially available protocols and scanners (Affymetrix, Inc., Santa Clara, Calif.). Subsequent analysis can, for example, consider each probe set as a separate gene. Expression values for each gene are calculated, for example, using Affymetrix GeneChip™ analysis software. Such analysis can optionally include quality control for the quality and/or quantity of the RNA as determined by, for example, optical density measurements and agarose gel electrophoresis. Threshold limits can be set according to the practitioner, but scans are preferably rejected if mean chip intensity exceeds 2 standard deviations from the average mean intensity for the entire scan set, if the proportion of “Present” calls is less than 10%, or if microarray artifacts are visible.

Genes that correlate with each tumor class can be identified by sorting all of the genes on the array according to their signal-to-noise values ((μ₀−μ₁)/(σ₀+σ₁), where μ and σ represent the mean and standard deviation of expression, respectively, for each class). For example, in one embodiment, one thousand permutations of the sample labels are performed on the dataset, and the signal-to-noise (S2N) ratio is recalculated for each gene for each class label permutations. A gene is considered a statistically significant class-specific marker if the observed S2N exceeded the permutated S2N at least 99% of the time (p<0.01).

The dataset is analyzed according to methods described herein. Using novel analytical methods, multi-class cancer classification and biological classification is indeed possible using a large database comprising expression data from several markers. This determination suggests the feasibility of molecular cancer diagnosis or diagnosis of other biological conditions with references to a comprehensive, commonly accessible catalog of expression data. For example, an expression database from 307 common human cancerous and normal tissues using oligonucleotide microarrays was established, as described in the examples, and the feasibility of cancer diagnosis by comparison of an unknown sample to this reference database was demonstrated.

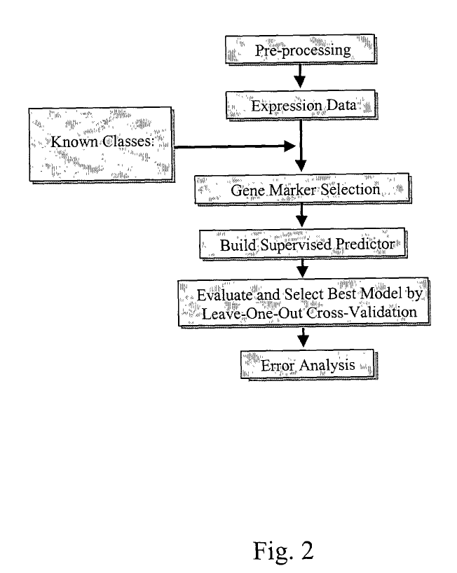

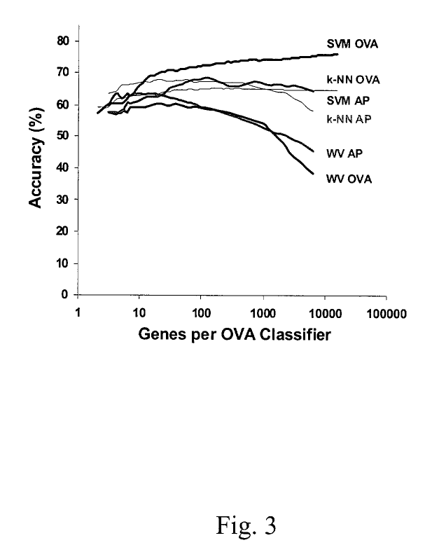

The dataset is preferably manipulated using a supervised learning algorithm (see FIG. 2) because this class of algorithms was found to more accurately predict tumor class (FIG. 3 and Examples). Supervised learning involves “training” a classifier to recognize distinctions among, for example, the 14 clinically-defined tumor classes in the dataset described in the Exemplification, based on gene expression patterns, and then testing the accuracy of the classifier in a blinded fashion. The methodology for building a supervised classifier differs from the algorithm used for predicting informative genes. In one embodiment, the algorithm models the dataset to allow for a series of pairwise One Versus All other (OVA) comparisons. The algorithm can be, for example, a linear or non-linear support vector machine (SVM) algorithm. For example, a linear SVM algorithm has strong theoretical foundations (Mukherjee, S. et al., Technical Report CBCL Paper 182/AI Memo 1676 MIT; Brown, M. et al., 2000. Proc. Natl. Acad. Sci. USA. 97:262-267; Furey, T. et al., 2000. Bioinformatics. 16:906-914; Vapnik, V., 1998. in Statistical Learning Theory. John Wiley & Sons, New York, N.Y.).

Multi-class predictions are intrinsically more difficult than binary prediction because the classification algorithm has to “learn” to construct a greater number of separation boundaries or relations. In binary classification an algorithm can “carve out” the appropriate decision boundary for only one of the classes; the other class is simply the complement. In multi-class classification each class has to be explicitly defined. Errors can occur in the construction of any one of the many decision boundaries, so the error rates on multi-class problems can be significantly greater than those of binary problems. For example, in contrast to a balanced binary problem where the accuracy of a random prediction is 50%, for K classes the accuracy of a random predictor is of the order of 1/K.

There are typically two types of multi-class classification algorithms. The first type deals directly with multiple values in the target field. For example Naïve Bayes, k-Nearest Neighbors, and classification trees are in this class. Intuitively, these methods can be interpreted as trying to construct a conditional density for each class, then classifying by selecting the class with maximum a posteriori probability. The second type decomposes the multi-class problem into a set of binary problems and then combines them to make a final multi-class prediction. This group contains support vector machines, boosting, and weighted voting algorithms, and, more generally, any binary classifier.

The basic idea behind combining binary classifiers is to decompose the multi-class problem into a set of easier and more accessible binary problems. The main advantage in this “divide-and conquer” strategy is that any binary classification algorithm can be used. Besides choosing a decomposition scheme and a base classifier, one also needs to devise a strategy for combining the binary classifiers and providing a final prediction. The problem of combining binary classifiers has been studied in the computer science literature (Hastie, T. and Tibshirani, R., 1998. Advances in Neural Processing Systems 10, MIT Press, Cambridge, Mass.; Guruswami, V. and Sahai, A., 1999. Proceedings of the Twelfth Annual Conference on Computational Learning Theory, ACM Press, 145-155) from a theoretical and empirical perspective. However, the literature is inconclusive with regard to the and the best method for combining binary classifiers for any particular problem is open.

Standard modern approaches to combining binary classifiers can be stated in terms of “output coding” (Dietterich and Bakiri, 1991. Proc. AAAI. 572-577). The concept of output coding is that given K classifiers trained on various partitions of the classes, a new example is mapped into an output vector. Each element in the output vector is the output from one of the K classifiers, and a “codebook” is then used to map from this vector to the class label. For example, given three classes, the first classifier can be trained to partition classes one and two from three, the second classifier trained to partition classes two and three from one, and the third classifier trained to partition classes one and two from three.

Two examples of output coding are the one-versus-all (OVA) and all-pairs (AP) approaches. In the OVA approach, given K classes, K independent classifiers are constructed where the ith classifier is trained to separate samples belonging to class i from all others. The codebook is a diagonal matrix, and the final prediction is based on the classifier that produces the strongest confidence,

$$class = arg {max}_{i = 1 … K} {f}_{i} ,$$

where fᵢ is the signed confidence measure of the ith classifier. In the AP approach, K(K−1)/2 classifiers are constructed with each classifier trained to discriminate between a class pair (i and j). This can be thought of as a K by K matrix, where the i-j th entry corresponds to a classifier that discriminates between classes i and j. The codebook in this case is used to simply sum the entries of each row and select the row for which this sum is maximum,

$$class = arg {max}_{i = 1 … K} [ {\sum }_{j = 1}^{K} {f}_{ij} ] ,$$

where as before fᵢⱼ is the signed confidence measure for the ijth classifier.

An ideal code matrix should be able to correct the mistakes made by the component binary classifiers. Dietterich and Bakiri used error-correcting codes to build the output code matrix where the final prediction is made by assigning a sample to the codeword with the smallest Hamming distance with respect to the binary prediction result vector (Dietterich and Bakiri, 1991. Proc. AAAI. 572-577). There are several other ways of constructing error-correcting codes including classifiers that learn arbitrary class splits and randomly generated matrices.

There is a tradeoff between the OVA and AP approaches. The discrimination surfaces that need to be learned in the AP approach are, in general, more natural and, theoretically, should be more accurate. However, with fewer training examples the empirical surface constructed may be less precise. The actual performance of each of these schemes, or others such as random codebooks, in combination with different classification algorithms is problem dependent.

Described herein is the use of Support Vector Machines (SVMs) in modeling datasets to allow for binary comparisons. The use of SVMs is provided as a non-limiting example. SVMs are powerful classification systems based on a variation of regularization techniques for regression (Vapnik, V., 1998. in Statistical Learning Theory. John Wiley & Sons, New York, N.Y.; Evgeniou, T. et al., 2000. Advances in Computational Mathematics, 13, 1-50). SVMs provide state-of-the-art performance in many practical binary classification problems. SVMs have also shown promise in a variety of biological classification tasks including some involving gene expression microarrays (Brown, M. et al., 2000. Proc. Natl Acad. Sci. USA. 97:262-267).

In a particular embodiment, the algorithm is a particular instantiation of regularization for binary classification. Linear SVMs can be viewed as a regularized version of a much older machine-learning algorithm, the perceptron (Rosenblatt, 1962. Principles of Neurodynamics. Spartan Books, New York, N.Y.; Minsky, M. and Papert, S., 1972. Perceptrons: An introduction to computational geometry. MIT Press, Cambridge, Mass.). The goal of a perceptron is to find a separating hyperplane that separates positive from negative examples. In general, there may be many separating hyperplanes. This separating hyperplane is the boundary that separates a given tumor class from the rest (OVA) or two different tumor classes (AP). The SVM chooses a separating hyperplane that has maximal margin, the distance from the hyperplane to the nearest point. Training an SVM requires solving a convex quadratic program with as many variables as training points.

SVMs assume the target values are binary and that the classification problem is intrinsically binary. The OVA methodology was used to combine binary SVM classifiers into a multi-class classifier. A separate SVM is trained for each class and the winning class is the one for with the largest margin, which can be thought of as a signed confidence measure.

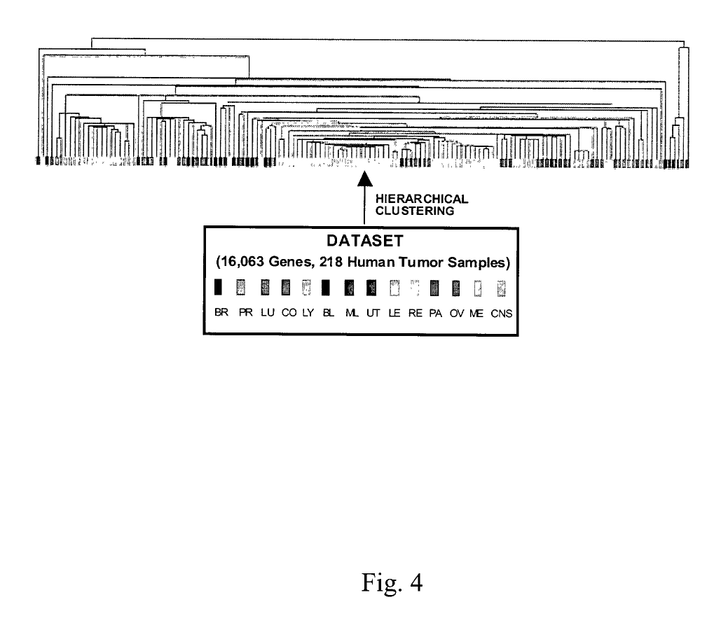

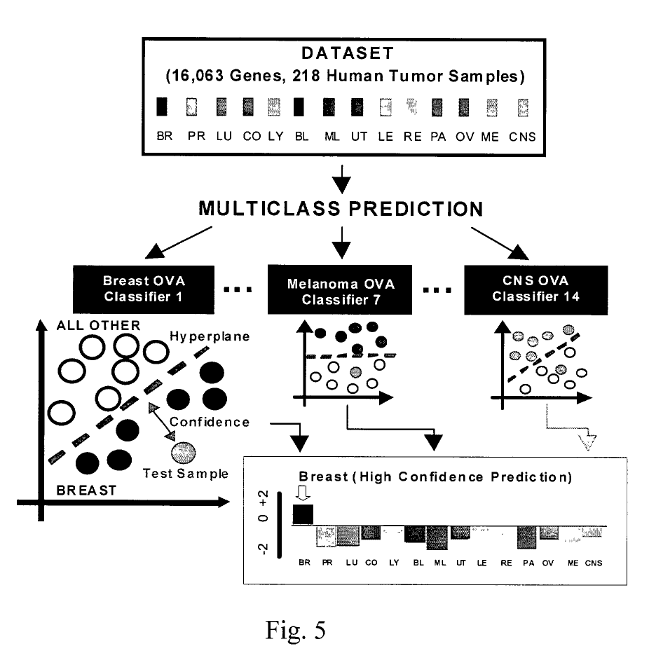

The SVM algorithm described herein can be, for example, a modified version of SvnFu (available the world wide web site: ai.mit.edu/projects/cbcl). This linear SVM algorithm, although non-linear SVM algorithms can also be used, defines a hyperplane that best separates tumor samples from two classes. In a particular case involving typical microarrays arranged on gene chips, the hyperplane is defined in 16,063-dimensional gene space (the total number of expression values considered; FIGS. 4 and 5). The SVM chooses the separating hyperplane with maximal margin, the distance from the hyperplane to the nearest point. An unknown test sample's position relative to the hyperplane determines its class and the confidence of each SVM prediction is based on the distance of a test sample from the hyperplane. In the one class versus all other classes (OVA) pairwise comparison scheme, a positive prediction strength corresponded to a test sample being assigned to the single class rather than to the “all other” class.

To determine a confidence level as to the predictive value of the methods described herein, a class-proportional random predictor can be used to determine the number of correct classifications that would be expected by chance for multi-class prediction. An associated p-value, the calculation of which is known to one of ordinary skill in the art, is calculated based on the likelihood that the observed classification accuracy could be arrived at by chance.

The decomposition of the multi-class classification into a series of binary comparisons allows for the accurate diagnosis of particular classes based on the information contained in large datasets. Manipulation of the datasets by, for example, SVMs into information suitable for use in a series of binary comparisons (e.g., OVA or AP comparisons), allows for the implementation of this approach. The promise of this approach lies in the fact that an extensive number of data points are used to train algorithms in allowing for the series of binary comparisons. Thus, accuracy increases as the size of the databases increases.

Expression-based cancer classification can be used in combination with more traditional diagnostic methods to further improve the accuracy of the diagnosis. Molecular characteristics of a tumor sample can remain intact despite atypical clinical or histologic features. All samples can be evaluated by a uniform method that can be standardized throughout the medical community. In addition, classification occurs through an algorithmic, rather than subjective approach in which classification confidence is quantified. A centralized classification database will allow classification accuracy to rapidly improve as the classification algorithm “learns” from an ever-growing database. As robust gene expression-based molecular correlates of stage, natural history, and treatment response are discovered, incorporation of this knowledge into the database will result in continually increasing clinical utility (Scherf, U. et al., 2000. Nat. Genet. 24:236-244; Kudoh, K. et al., 2000. Cancer Res. 60:4161-4166).

The 14-tumor type classifier described in the Exemplification was demonstrated to be more accurate than other methods, and error values were assigned to predict a degree of confidence in the accuracy of the classification. The distribution of errors throughout the solid tumor classes implies that improved accuracy is possible by increasing the number of samples in the training set, beyond the modest number used here (on average, 10 per class). In addition, the classification strategy used could vary slightly for every type of multi-class classification problem. Other classification schemes, classification algorithms, or novel marker selection methods can also be useful for making multi-class distinctions (Hastie, T. et al., 2000. Genome Biol. 1:research003.1-0003.21; Tusher, V. et al., 2001. Proc. Natl. Acad. Sci. USA. 98:5116-5121; Alter, O. et al., 2000. Proc. Natl. Acad. Sci. USA. 97:10101-10106; Kim, S. et al., 2000. Genomics. 67:201-209). For example, one might use a decision tree wherein the cancer versus normal distinction is made, followed by site of origin classification and further sub-typing.

The invention will be further described with reference to the following non-limiting examples. The teachings of all the patents, patent applications and all other publications and websites cited herein are incorporated by reference in their entirety.

### EXEMPLIFICATION

### Example 1

### An Approach to Molecular Cancer Diagnosis Using a Trained Algorithm

Materials and Methods

Snap-frozen human tumor and normal tissue specimens, spanning 14 different tumor classes, were obtained from the NCI/Cooperative Human Tissue Network, the Massachusetts General Hospital Tumor Bank, and individual investigators at the Dana-Farber Cancer Institute, Brigham and Women's Hospital, Children's Hospital-Boston, Memorial Sloan-Kettering Cancer Center, and Biochain, Inc. (Hayward, Calif.). Three classes contained known cancer subtypes: lymphoma (large B-cell, follicular), leukemia (acute myelogenous, acute lymphocytic (B-cell and T-cell)), and central nervous system tumors (medulloblastoma, glioblastoma). The tumors were biopsy specimens obtained prior to any treatment. All tumors underwent centralized pathology review at the Dana-Farber Cancer Institute and Brigham and Women's Hospital, Children's Hospital-Boston, or Memorial Sloan-Kettering Cancer Center, and were collected in an anonymous fashion under a discarded tissue protocol approved by the Dana-Farber Cancer Institute Institutional Review Board.

RNA from whole tumors was used to prepare “hybridization targets” according to published methods (Golub, T. et al., 1999. Science. 286:531-537). Targets were hybridized sequentially to oligonucleotide microarrays containing a total of 16,063 probe sets representing 14,030 GenBank and 475 TIGR accession numbers. Affymetrix Hu6800 and Hu35KsubA GeneChips™ and arrays were scanned using standard Affymetrix protocols and scanners. For subsequent analysis, each probe set was considered as a separate gene. Expression values for each gene were calculated using Affymetrix GeneChip™ analysis software. Of 314 tumor samples and 98 normal tissue samples processed, 217 tumors and 90 normal tissue samples passed quality control criteria and were used for subsequent data analysis. The remaining 105 samples either failed quality control measures of the amount and quality of RNA, as assessed by spectrophotometric measurement due to optical density (OD) and agarose gel electrophoresis, or yielded poor quality scans. Scans were rejected if mean chip intensity exceeded 2 standard deviations from the average mean intensity for the entire scan set, if the proportion of “Present” calls was less than 10%, or if microarray artifacts were visible.

Clustering. Gene expression data were subjected to a variation filter that excluded genes showing minimal variation across the samples being analyzed. Clustering was performed following exclusion of genes with less than 5-fold and 500 units absolute variation across the dataset after a threshold of 20 units and ceiling of 16,000 was applied. Of 16,063 expression values considered, 11,322 passed this filter and were used for clustering. The dataset was normalized by standardizing each column (sample) to mean=0 and variance=1. Hierarchical clustering was performed using Cluster and TreeView software (Eisen, M. et al., 1998. Proc. Natl. Acad. Sci. USA., 95:14863-14868; FIG. 4). Self-organizing maps (SOMs) analysis was performed using the GeneCluster analysis package.

Results

Expression data of 144 primary tumors was obtained using oligonucleotide microarrays containing 16,063 oligonucleotide probe sets. Centralized histological review was used to confirm each clinical diagnosis. All tumors in this set were enriched in malignant cells but otherwise unselected. Tumor samples were primarily solid tumors of epithelial origin, spanning 14 common tumor classes that account for approximately 80% of new cancer diagnoses in the United States.

Two fundamentally different approaches to data analysis were explored. The first, unsupervised learning, often referred to as clustering, allows the dominant molecular structure in a dataset to dictate the separation of samples into clusters based on overall similarity in gene expression, without prior knowledge of sample identity. FIG. 4 shows the result of hierarchical clustering of this dataset. While some tumor types such as lymphoma, leukemia, and central nervous system tumors formed relatively discrete clusters, others, in particular the epithelial tumors, were largely scattered among the branches of the dendrogram. Similar results were obtained with an alternative clustering algorithm, SOMs. These findings indicate that unsupervised learning methods do not adequately capture the tissue of origin distinctions among these molecularly complex tumors. The hierarchical tree structure might reflect bonafide, previously unrecognized relationships among tumors that transcend tissue of origin distinctions.

The second approach used to address this classification problem involved using supervised machine learning methods, which in this particular case involved “training” a classifier to recognize the distinctions among the 14 clinically-defined tumor classes based on gene expression patterns, and then testing the accuracy of the classifier in a blinded fashion. Supervised learning has been used to generate models used in making pairwise distinctions with gene expression data (e.g., the distinction between acute lymphoblastic leukemia (ALL) and acute mycloid leukemia (AML); Golub, T. et al., 1999. Science. 286:531-537), but making multi-class distinctions is a considerably more difficult challenge (Khan, J. et al., 2001. Nat. Med. 7:673-679). For this purpose, a novel analytical scheme, depicted in FIG. 2, was devised. First, the multi-class problem was divided into a series of 14 one class versus all other classes (OVA) pairwise comparisons. Each test sample was presented sequentially to 14 pairwise classifiers, each of which either claimed or rejected that sample as belonging to the class. This resulted in 14 separate OVA classifications per sample, each with an associated confidence. Each test sample was assigned to the class with the highest OVA classifier confidence.

Several classification algorithms for these OVA pairwise classifiers including Weighted Voting (Slonim, D., 2000. in Proceedings of the Fourth Annual International Conference on Computational Molecular Biology (RECOMB). Universal Academy Press, Tokyo, Japan, pp. 263-272), k-Nearest Neighbors (Dasarathy, V. (ed), 1991. in Nearest Neighbor (NN) Norms NN PAttern Classification Techniques. IEEE Computer Society Press. Los Alamitos, Calif.) and Support Vector Machines were evaluated. The SVM algorithm performed with greatest overall accuracy, and these results are described. The SVM algorithm considers all profiled markers and defines a hyperplane that best separates tumor samples from two classes (FIG. 5). An unknown sample's position relative to this hyperplane determines its membership in one or other class (e.g., ‘breast cancer’ versus ‘not breast cancer’). 14 separate OVA classifiers classify each sample. The confidence of each OVA SVM prediction is based on the distance of the test sample to each hyperplane, with a value of 0 indicating that a sample falls on a hyperplane. The classifier then assigns a sample to the class with the highest confidence among the 14 pairwise OVA analyses.

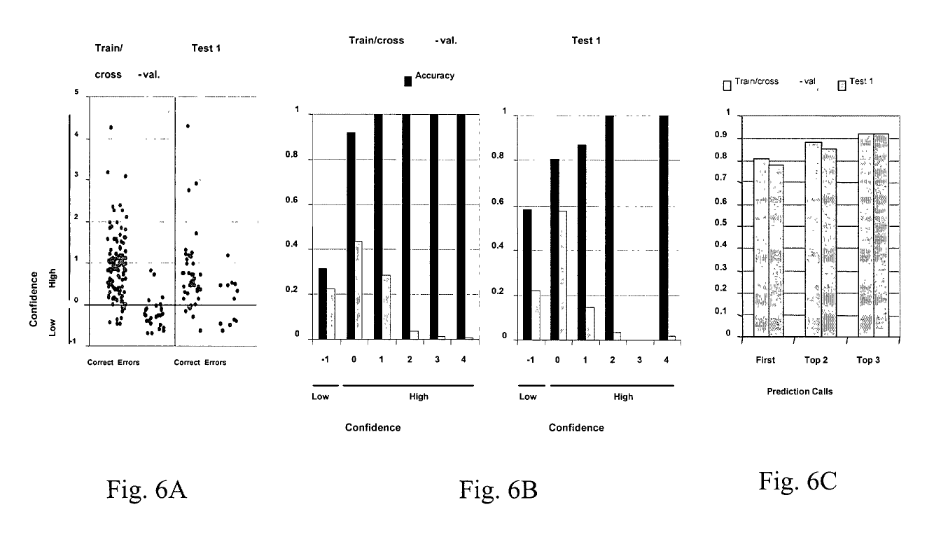

The accuracy of this multi-class SVM-based classifier in cancer diagnosis was evaluated by cross-validation. This method involves randomly withholding one of the 144 tumor samples, building a predictor based only on the remaining samples, and then predicting the class of the withheld sample. The process is repeated for each sample and the cumulative error rate is calculated. As shown in FIG. 6, the majority (76%) of the 144 calls were high confidence (defined as confidence>0) and these had an accuracy of 96%. The remaining 24% of the tumors had low confidence calls (confidence≦0) and these predictions had an accuracy of 32%. Overall, the multi-class prediction corresponded to the correct assignment for 81% of the tumors; this is substantially higher than the expected result of 9% for random prediction in this fourteen-class problem. For an additional 11% of the tumors, which were incorrectly classified, the correct answer corresponded to the second- or third-most confident OVA prediction. These results demonstrate that uniform and comprehensive molecular cancer classification is possible using whole tumor gene expression profiles.

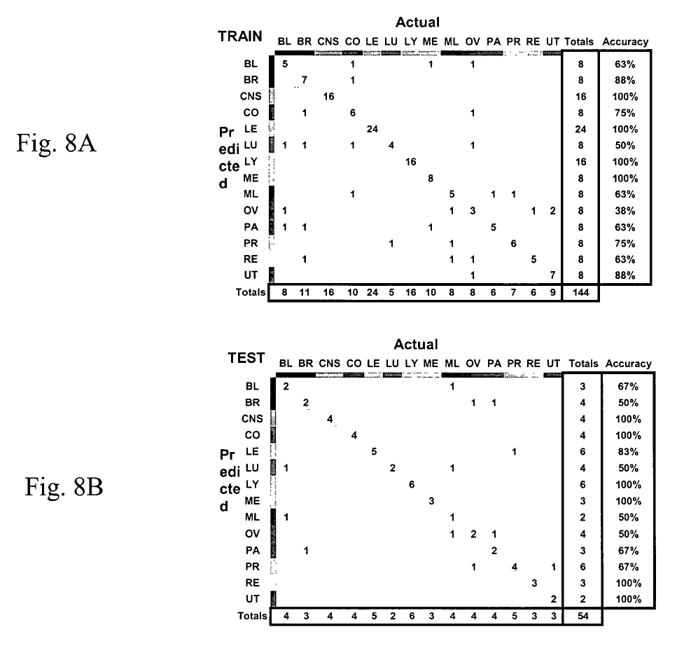

This result was confirmed by training the multi-class SVM classifier on the entire set of 144 samples and applying this classifier to an independent test set of 54 tumor samples. Overall prediction accuracy on this test set was 78%, a result similar to cross-validation accuracy and highly statistically significant when compared with random prediction (p≦10⁻¹⁶). The majority (78%) of these 54 predictions were again high confidence, and had an accuracy of 83%, whereas low confidence calls were made on the remaining 22% of tumors with an accuracy of 58%. For an additional 11% of the cases, the correct answer corresponded to the second- or third-best prediction. Of note, classification of 100 random splits of a combined training and test dataset gave similar results, confirming the stability of the predictor for this collection of samples (FIGS. 8A and 8B). Significantly, among these 54 test samples were 8 metastatic samples, 6 of which were correctly classified despite the classifier having been trained solely with gene expression data derived from primary tumors (p=0.005 compared to random classification). This finding implies that prediction is being driven by tumor-intrinsic gene expression patterns rather than by gene expression signatures derived from contaminating normal tissue elements. These results further indicate that many cancer types retain their tissue of origin identity throughout metastatic evolution, suggesting that gene expression-based approaches to the diagnosis of clinically problematic metastases of unknown primary origin could be feasible (Hainsworth, J. and Greco, F., 1993. N. Engl. J Med. 329:257-263).

The number of genes contributing to the high accuracy of the SVM classifier was investigated next. The SVM algorithm utilized all 16,063 input genes, each of which is assigned a weight based on its relative contribution to the determination of each OVA classification hyperplane. Markers that do not contribute to a distinction are given a weight of zero. Virtually all of genes on the array were assigned weakly positive and negative weights in each OVA classifier, indicating that thousands of genes carry information that is relevant for the 14 OVA class distinctions. To determine whether the inclusion of this large number of genes was actually required for the observed high accuracy predictions, the relationship between classification accuracy and marker number was determined. As shown in FIGS. 8A and 8B, classification accuracy falls significantly as the predictor utilizes fewer markers. This finding implies that information useful for multi-class tumor classification is encoded in complex gene expression patterns rather than in a small number of discrete genes. Alternatively, the use of thousands of markers might serve a smoothing function to counteract intrinsic noise in this expression dataset. This observation has significant implications for the future clinical implementation of molecular diagnostic approaches to cancer.

This expression dataset is also useful for biomedical discovery. For example, many of the markers most highly correlated with each of the 14 tumor classes lack individual statistical significance, as measured by the class-label permutation test. Nevertheless, many markers already in routine clinical use for cancer diagnosis were revealed in the study, including prostate specific antigen (prostate cancer), carcinoembryonic antigen (colon cancer), CD20 (lymphoid cancers), S100 (melanoma) and estrogen receptor (uterine cancer). In addition, many previously unrecognized markers were discovered, the vast majority of which are tissue-specific genes, reflecting the significant degree of shared gene expression between tumors and their normal tissue counterparts. Despite this overlapping gene expression, however, 209 primary tumors considered as a single class could still be distinguished from a collection of 90 normal tissues with high accuracy (92%), indicating the presence of a cancer-specific gene expression footprint common to all tumors.

Of the markers most highly correlated with the distinction of one tumor type versus all others, many are expressed during normal organ development, reflecting a recurring onco-developmental connection that has been described for several cancers (Taipale, J. and Beachy, P., 2001. Nature. 411:349-354). For example, a search for colorectal adenocarcinoma-specific markers revealed 27 that were statistically significant (p<0.01 based on random permutation testing). This set of markers includes intestine-specific transcription factors, cytoskeletal and adhesion molecules, signaling molecules, and membrane-bound tumor markers. Notably, the two transcription factors, Cdx-1 and Bteb-2, are both targets of the Wnt-1/β-Catenin signaling pathway that is mutated in nearly all colorectal cancers (Lickert, H. et al., 2000. Development. 127:3805-3813; Ziemer, L. et al., 2001. Mol. Cell. Biol. 21:562-574; Bienz, M. and Clevers, H., 2000. Cell. 103:311-320). The other colon cancer markers are thus also candidates for being under Wnt-1/β-Catenin control. This observation suggests that the expression database described herein will be useful not only for cancer diagnosis, but also for the generation of new biological hypotheses into the pathogenesis of cancer.

The samples that yielded low confidence predictions were also examined since these samples were generally mis-classified by the multi-class predictor. A large number (15 of 27 errors in cross-validation) of these samples included modestly to poorly differentiated (high-grade) carcinomas. Classifying such tumors can be difficult using traditional methods because they often lack the characteristic morphological hallmarks of the organ from which they arise. It has been assumed that these tumors are nonetheless fundamentally molecularly similar to their better-differentiated counterparts, apart from a few differences that might account for their clinically aggressive nature. This hypothesis was tested directly by applying the multi-class classifier, trained on the original 144 tumor dataset, to an independent set of poorly differentiated tumors.

Expression data was collected on 20 poorly differentiated adenocarcinomas (14 primary and 6 metastatic), representing 4 tumor types: breast, lung, colon, and ovary. The technical quality of this dataset was indistinguishable from the other samples in the study. These tumors could not be accurately classified according to their tissue of origin. Overall only 6/20 samples (30%) were correctly classified, which is statistically no better than what one would expect by chance alone (p=0.38). Because the classifier relies on the expression of thousands of similarly weighted, tissue-specific molecular markers to determine the class of a tumor, these findings indicate that, surprisingly, the expression patterns of poorly differentiated tumors have not simply lost some key markers of differentiation, but are fundamentally different. This result has significant implications for the future management of patients with these cancers. Given the clinically aggressive nature of poorly differentiated cancers, some of the markers that are preferentially expressed by poorly differentiated tumors might prove generally useful for predicting poorer clinical outcome.

### Example 2

### Multi-class Cancer Diagnosis Using Tumor Gene Expression Signatures

Materials and Methods

The gene expression datasets were obtained following an experimental protocol shown schematically in FIG. 1. Initial diagnoses were made at university hospital referral centers using all available clinical and histopathologic information. Tissues underwent centralized clinical and pathology review at the Dana-Farber Cancer Institute and Brigham & Women's Hospital or Memorial Sloan-Kettering Cancer Center to confirm initial diagnosis of site of origin. All tumors were:

1. biopsy specimens from primary sites (except where noted)

2. obtained prior to any treatment

3. enriched in malignant cells (>50%) but otherwise unselected.

Normal tissue RNA (Biochain, Inc. (Hayward, Calif.)) was from snap-frozen autopsy specimens collected through the International Tissue Collection Network.

Microarray hybridization. RNA from whole tumors was used to prepare “hybridization targets” with previously published methods. Briefly, snap frozen tumor specimens were homogenized (Polytron, Kinematica, Lucerne) directly in Trizol (Life Technologies, Gaithersberg, Md.), followed by a standard RNA isolation according to the manufacturer's instructions. RNA integrity was assessed by non-denaturing gel electrophoresis (1% agarose) and spectrophotometry. The amount of starting total RNA for each reaction was 10 μg. First strand cDNA synthesis was performed using a T7-linked oligo-dT primer, followed by second strand synthesis. An in vitro transcription reaction was performed to generate cRNA containing biotinylated UTP and CTP, which was subsequently chemically fragmented at 95° C. for 35 minutes. Fifteen micrograms of the fragmented, biotinylated cRNA was sequentially hybridized in MES buffer (2-[N-Morpholino]ethansulfonic acid) containing 0.5 mg/mL acetylated bovine serum albumin (Sigma, St. Louis, Mo.) to Affymetrix (Santa Clara, Calif.) Hu6800 and Hu35KsubA oligonucleotide microarrays at 45° C. for 16 hours. Arrays were washed and stained with streptavidin-phycoerythrin (SAPE, Molecular Probes, Eugene, Oreg.).

Signal amplification was performed using a biotinylated anti-streptavidin antibody (Vector Laboratories, Burlingame, Calif.) at 3 μg/mL followed by a second staining with SAPE. Normal goat IgG (2 mg/mL) was used as a blocking agent.

Scans were performed on Affymetrix scanners and expression values for each gene was calculated using Affymetrix GeneChip™ software. Hu6800 and Hu35KsubA arrays contain a total of 16,063 probe sets representing 14,030 GenBank and 475 TIGR accession numbers. For subsequent analysis, the output of each probe set (e.g., the “average difference” value calculated from matched and mismatched probe hybridization) was considered as a separate gene.

Of 314 tumor samples and 98 normal tissue samples processed, 218 tumors and 90 normal tissue samples passed quality control criteria and were used for subsequent data analysis. The remaining 104 samples either failed quality control measures of the amount and quality of RNA, as assessed by spectrophotometric measurement of optical density (OD) and agarose gel electrophoresis, or yielded poor quality scans. Scans were rejected if mean chip intensity exceeded 2 standard deviations from the average mean intensity for the entire scan set, if the proportion of “Present” calls was less than 10%, or if microarray artifacts were visible. This resulting dataset has approximately 5 million gene expression values.

Data (308 samples) was organized into four sets:

1. GCM_Training.res (Training Set; 144 primary tumor samples)

2. GCM_Test.res (Independent Test Set; 54 samples; 46 primary and 8 metastatic)

3. GCM_PD.res (Poorly differentiated adenocarcinomas; 20 samples)

4. GCM_All.res (Training set+Test set+normals (90); 280 samples).

In each dataset, columns represent each gene profiled, rows represent samples, and the values are raw average difference value output from the Affymetrix software package.

Support Vector Machines. Support Vector Machines (SVMs) are powerful classification systems based on a variation of regularization techniques for regression (Vapnik, V., 1998. in Statistical Learning Theory. John Wiley & Sons, New York, N.Y.; Evgeniou, T. et al., 2000. Advances in Computational Mathematics, 13, 1-50). SVMs provide state-of-the-art performance in many practical binary classification problems. SVMs have also shown promise in a variety of biological classification tasks including some involving gene expression microarrays (Brown, M. et al., 2000. Proc. Natl Acad. Sci. USA. 97:262-267).

The algorithm is a particular example of a regularization for binary classification. Linear SVMs can be viewed as a regularized version of a much older machine-learning algorithm, the perceptron (Rosenblatt, 1962. Principles of Neurodynamics. Spartan Books, New York, N.Y.; Minsky, M. and Papert, S., 1972. Perceptrons: An introduction to computational geometry. MIT Press, Cambridge, Mass.). The goal of a perceptron is to find a separating hyperplane that separates positive from negative examples. In general, there may be many separating hyperplanes. This separating hyperplane is the boundary that separates a given tumor class from the rest (OVA) or two different tumor classes (AP). The SVM chooses a separating hyperplane that has maximal margin, the distance from the hyperplane to the nearest point. Training an SVM requires solving a convex quadratic program with as many variables as training points.

SVMs assume the target values are binary and that the classification problem is intrinsically binary. The OVA methodology was used to combine binary SVM classifiers into a multi-class classifier. A separate SVM is trained for each class and the winning class is the one for with the largest margin, which can be thought of as a signed confidence measure.

In the experiments described herein, there are a few data points in many dimensions. Therefore, a linear classifier was used in the SVM. Although the hyperplane was not allowed to make misclassifications, in all cases involving the full 16,063 dimensions, each OVA hyperplane fully separated the training data with no errors. In some of the experiments involving explicit feature selection with very few features, there were some training errors. Although this could have indicated that a very small number of features could be selected followed by using a kernel function to improve classification, experiments with this approach yielded no improvement over the linear case.

Recursive Feature Elimination. Many methods exist for performing feature selection. Similar results were observed with informal experiments using recursive feature elimination (RFE), signal to noise ratio (Slonim, D., 2000. in Proceedings of the Fourth Annual International Conference on Computational Molecular Biology (RECOMB). Universal Academy Press, Tokyo, Japan, pp. 263-272), and the radius-margin-ratio (Weston et al., 2001). RFE was used since it is the most straightforward to implement with the SVM. The method recursively removes features based upon the absolute magnitude of the hyperplane elements. Given microarray data with n genes per sample, the SVM outputs a hyperplane, w, which can be thought of as a vector with n components each corresponding to the expression of a particular gene. Assuming that the expression values of each gene have similar ranges, the absolute magnitude of each element in w determines its importance in classifying a sample, since,

$$f ( x ) = {\sum }_{i = 1}^{n} {w}_{i} {x}_{i} + b ,$$

and the class label is [f(x)]. The SVM is trained with all genes, the expression values of genes corresponding to |wᵢ| in the bottom 10% are removed and the SVM is retrained with the smaller gene expression set.

Proportional chance criterion. In order to compute p-values for multi-class prediction, a “proportional chance criterion” was used to evaluate the probability that a random predictor will produce a confusion matrix with the same row and column counts as the gene expression predictor. For example, for a binary class (A vs. B) problem, if α is the prior probability of a sample being in class A and p is the -true proportion of samples in class A, then Cₚ=pα+(1−p) (1−α) is the proportion of the overall sample that is expected to receive correct classification by chance alone. Then if C_{model} is the proportion of correct classifications achieved by the gene expression predictor one can estimate its significance by using a Z statistic of the form: (C_{model}−Cₚ)/Sqrt(Cₚ(1−Cₚ)/n), where n is the total sample count. For more details see chapter VII of Huberty's Applied Discriminant Analysis (Huberty, C., 1994. Applied Discriminant Analysis. John Wiley & Sons, New York, N.Y.).

Multi-class Prediction Results. In a preliminary empirical study of multi-class methods and algorithms (Yeang, C. et al., 2001. Bioinformatics. 17(S1):s316-s322), the OVA and AP approaches were applied with three different algorithms: Weighted Voting, k-Nearest Neighbors and Support Vector Machines. The results, shown in Table 2, demonstrate that the OVA approach in combination with SVM provided the most accurate method by a significant margin.

SVM/OVA Multi-class Prediction. The procedure for this approach is as follows:

1) Define each target class based on histopathologic clinical evaluation (pathology review) of tumor specimens;

2) Decompose the multi-class problem into a series of 14 binary OVA classification problems: one for each class.

3) For each class optimize the binary classifiers on the training set using leave-one-out cross-validation (e.g., remove one sample, train the binary classifier on the remaining samples), combine the individual binary classifiers to predict the class of the left out sample, and iteratively repeat this process for all the samples. A cumulative error rate is calculated.

4) Evaluate the final prediction model on an independent test set.

This procedure is described pictorially in FIG. 5 where the bar graphs on the lower right side show an example of actual SVM output predictions for a Breast adenocarcinoma sample.

The final prediction (winning class) of the OVA set of classifiers is the one corresponding to the largest confidence (margin),

$$class = arg {max}_{i = 1 … K} {f}_{i} .$$

The confidence of the final call is the margin of the winning SVM. When the largest confidence is positive the final prediction is considered a “high confidence” call. If negative it is a “low confidence” call that can also be considered a candidate for a no-call because no single SVM “claims” the sample as belonging to its recognizable class. The error rates were analyzed in terms of totals and also in terms of high and low confidence calls. In the example in the lower right hand side of FIG. 5, an example of a high confidence call, the Breast classifier attains a large positive margin while the other classifiers all have negative margins.

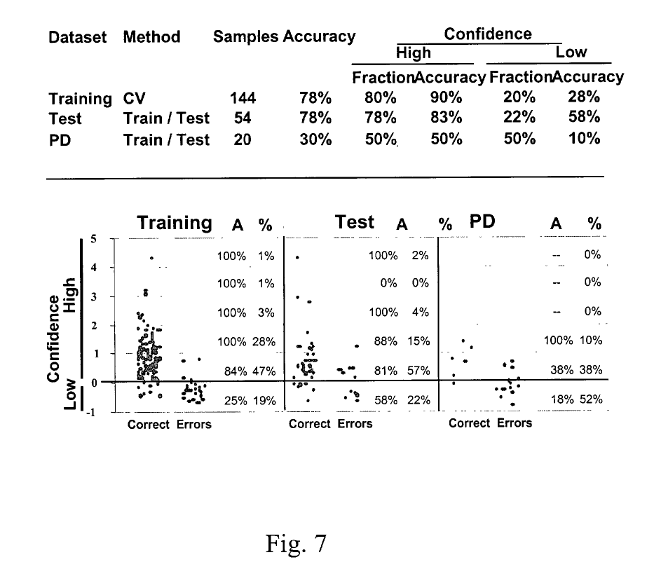

Repeating this procedure we created a multi-class OVA-SVM model with all genes using the training dataset and then applied it to two test datasets (Independent Test Set and Poorly differentiated adenocarcinomas). The results are summarized in FIG. 7 (Top).

As can be seen in the table in cross validation the overall multi-class predictions were correct for 78% of the tumors. This accuracy is substantially higher than expected for random prediction (9% according to proportional chance criterion). More interestingly the majority of calls (80%) were high confidence, and for these the classifier achieved an accuracy of 90%. The remaining tumors (20%) had low confidence calls and lower accuracy (28%). The results for the test set are similar to the ones obtained in cross-validation: the overall prediction accuracy was 78% and the majority of these predictions (78%) were again high confidence with an accuracy of 83%. Low confidence calls were made on the remaining 22% of tumors with an accuracy of 58%.

The actual confidences for each call and a bar graph of accuracy and fraction of calls versus confidence is shown in FIG. 7B. The confusion matrices for cross-validation (Train) and Independent Test Set (Test) are shown in FIGS. 8A and 8B.

An interesting observation concerning these results is that for 50% of the tumors that were incorrectly classified the correct answer corresponded to the second or third most confident (SVM) prediction (FIGS. 6A-6C).

To confirm the stability and reproducibility of the prediction results for this collection of samples, the train and test procedure for 100 random splits of a combined dataset were repeated. The results were similar to the reported case. FIG. 3 shows the mean of the error rate for the different test-train splits as a function of the total number of genes. Due to the fact the different test-train splits were obtained by reshuffling the dataset the empirical variance measured is optimistic (Efron, B. and Tibshirani, R., 1993. Introduction to the Bootstrap. Chapman and Hall, New York, N.Y.).

The accuracy of the multi-class SVM predictor as a function of the number of genes was also analyzed. The algorithm inputs all of the 16,063 genes in the array and each of them is assigned a weight based on its relative contribution to each OVA classification. Practically all genes were assigned weakly positive and negative weights in each OVA classifier. Multiple runs were performed with different numbers of genes selected using RFE. Results are also shown in FIG. 3, where total accuracy decreases as the number of input genes decreases for each OVA distinction. Pairwise distinctions can be made between some tumor classes using fewer genes but multi-class distinctions among highly related tumor types are intrinsically more difficult. This behavior can also be the result of the existence of molecularly distinct but unknown subclasses within known classes that effectively decrease the predictive power of the multi-class method. Despite the increasing accuracy with increased number of genes trend, significant but modest prediction accuracy can be achieved with a relatively small number of genes per classifier (e.g., about 70% with about 200 total genes).

Support Vector Machines. The problem of learning a classification boundary given positive and negative examples is a particular case of the problem of approximating a multivariate function from sparse data. The problem of approximating a function from sparse data is ill-posed and regularization theory is a classical approach to solving it (Tikhonov and Arsenin, 1977. Solutions of ill-posed problems, W. H. Winston, Washington, D.C.).

Standard regularization theory formulates the approximation problem as a variational problem of finding the function f that minimizes the functional

$${min}_{f ∈ H} \frac{1}{l} {\sum }_{l = 1}^{l} V ( {y}_{i} , f ( {x}_{i} ) ) + λ {‖ f ‖}_{K}^{2}$$

where V(,) is a loss function, ∥ƒ∥²_{K} is a norm in a Reproducing Kernel Hilbert Space defined by the positive function K (Aronszsajn 1950), l is the number of training examples, and λ is the regularization parameter. Under rather general conditions the solution to the above functional has the form

$$f ( x ) = {\sum }_{l = 1}^{l} {α}_{i} K ( x , {x}_{i} )$$

SVMs are a particular case of the above regularization framework (Evgeniou, T. et al., 2000. Advances in Computational Mathematics, 13, 1-50).

For the SVM the regularization functional minimized is the following

$${min}_{f ∈ H} \frac{1}{l} {\sum }_{l = 1}^{l} {( 1 - {y}_{i} f ( {x}_{i} ) )}_{+} + λ {‖ f ‖}_{K}^{2} ,$$

where the hinge loss function is used, (a)₊ is the min(a,0). The solution again has the form

$$f ( x ) = {\sum }_{l = 1}^{l} {α}_{i} K ( x , {x}_{i} ) . ,$$

and the label output is simply sign(ƒ(x)).

The SVM an also be developed using a geometric approach. A hyperplane is defined via its normal vector w. Given a hyperplane w and a point x, define x₀ to be the closest point to x on the hyperplane- the closest point to x that satisfies w·x₀=0. The following two equations result:

w·x=k for some k

w·x₀ =0.

Subtracting these two equations,

w·(x−x₀)=k

Dividing by the norm of w,

$$\frac{w}{‖ w ‖} \cdot ( x - {x}_{0} ) = \frac{k}{‖ w ‖} .$$

Noting that

$$\frac{w}{‖ w ‖}$$

is a unit vector, and the vector x−x₀ is parallel to w, the conclusion is,

$$‖ x - {x}_{0} ‖ = \frac{\vert k \vert}{‖ w ‖} .$$

The goal is to maximize the distance between the hyperplane and the closest point, with the constraint that the points from the two classes lie on separate sides of the hyperplane. In trying to solve the following optimization problem:

$${max}_{w} {min}_{{x}_{i}} \frac{{y}_{i} ( w \cdot {x}_{i} )}{‖ w ‖} ,$$

subject to yᵢ(w·xᵢ)>0 for all xᵢ.

Note that yᵢ(w·xᵢ)=|k| in the above derivation. For technical reasons, the optimization problem stated above is not easy to solve. One difficulty is that if a solution w is found, then cw for any positive constant c is also a solution. In some sense, the direction of the vector w, but not its length, is of interest.

If any solution w to the above problem can be found, for example by scaling w, it can be guarantee that yᵢ(w·xᵢ)≧1 for all xᵢ. Therefore, the problem is equivalently solved,

$${max}_{w} {min}_{{x}_{i}} \frac{{y}_{i} ( w \cdot {x}_{i} )}{‖ w ‖} subject to {y}_{i} ( w \cdot {x}_{i} ) \geq 1.$$

Note that the original problem has more solutions than this one, but since the only interest is in the direction of the optimal hyperplane, this would suffice. Restricting the problem further, a solution will be found such that for any point closest to the hyperplane, the inequality constraint will be satisfied as an equality. Keeping this in mind,

$${min}_{{x}_{i}} \frac{{y}_{i} ( w \cdot {x}_{i} )}{‖ w ‖} = 1.$$

So the problem becomes,

$$max \frac{1}{‖ w ‖}$$

subject to yᵢ(w·xᵢ)≧1

For computational reasons, the problem is transformed to the equivalent problem,

$$min \frac{1}{2} {‖ w ‖}^{2}$$

subject to yᵢ(w·xᵢ)≧1.

Note that so far, only hyperplanes have been considered that pass through the origin. In many applications, this restriction is unnecessary, and the standard separable SVM problem is written as,

$$min \frac{1}{2} {‖ w ‖}^{2} subject to {y}_{i} ( w \cdot {x}_{i} + b ) \geq 1$$

where b is a free threshold parameter that translates the optimal hyperplane away from the origin.

In practice, datasets are often not linearly separable. To deal with this situation, slack variables are added that allow one to violate the original distance constraints. The problem becomes:

$$min \frac{1}{2} {‖ w ‖}^{2} + C {\sum }_{i}^{} {χ}_{i}$$

subject to yᵢ(w·xᵢ+b)≧1−xᵢ,xᵢ≧0 for all i.

This new program trades off the two goals of finding a hyperplane with large margin (minimizing ∥w∥), and finding a hyperplane that separates the data well (minimizing the xᵢ). The parameter C controls this tradeoff. It is no longer simple to interpret the final solution of the SVM problem geometrically; however, this formulation often works very well in practice. Even if the data at hand can be separated completely, it could be preferable to use a hyperplane that makes some errors, if this results in a much smaller ∥w∥.

There also exist SVMs that can find a nonlinear separating surface. The basic idea is to nonlinearly map the data to a feature space of high or possibly infinite dimension,

x→φ(x).

A linear separating hyperplane in the feature space corresponds to a nonlinear surface in the original space. The program can be written as follows,

$$min \frac{1}{2} {‖ w ‖}^{2} + C {\sum }_{i}^{} {χ}_{i}$$

subject to, yᵢ(w·φxᵢ+b)≧1−xᵢ, xᵢ≧0 for all i.

Note that as phrased above, w is a hyperplane in the feature space. In practice, the Wolfe dual of the optimization problems presented is solved. A nice consequence of this is that there is no need to work with w and φ(x), the hyperplane and the feature vectors, explicitly. Instead, only a function, K(x,y) is needed that acts as a dot product in feature space,

K(xᵢ, xⱼ)=φ(xᵢ)·φ(xⱼ).

For example, if a Gaussian kernel is used as the kernal function,

K(xᵢ, xⱼ)=exp (−∥xᵢ−xⱼ∥²).

This corresponds to mapping the original vectors xᵢ to a certain countably infinite dimensional feature space when x is in a bounded domain and an uncountably infinite dimensional feature space when the domain is not bounded.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of classifying a biological sample comprising:
determining the expression pattern of one or more markers in a biological sample;
providing a model generated by a supervised learning algorithm based on a dataset of expression values from known biological classes; and
comparing the expression pattern of the markers in the sample to the model,
thereby classifying said biological sample.

2. The method of claim 1, wherein the biological sample is classified as a disease sample or as a normal sample.

3. The method of claim 2, wherein the disease is selected from the group consisting of cancer, coronary artery disease, neurodegenerative disease and pulmonary disease.

4. The method of claim 3, wherein the disease is cancer.

5. The method of claim 1, wherein the dataset comprises data from known classes of a particular disease.

6. The method of claim 5, wherein the particular disease is selected from the group consisting of cancer, coronary artery disease, neurodegenerative disease and pulmonary disease.

7. The method of claim 6, wherein the disease is cancer.

8. The method of claim 7, wherein the classes of cancer are selected from the group consisting of breast adenocarcinoma, prostate adenocarcinoma, lung adenocarcinoma, colorectal adenocarcinoma, lymphoma, bladder transitional cell carcinoma, melanoma, uterine adenocarcinoma, leukemia, renal cell carcinoma, pancreatic adenocarcinoma, ovarian carcinoma, pleural mesothelioma and central nervous system.

9. The method of claim 1, wherein the biological sample is compared to the model in a pairwise manner for each biological class.

10. The method of claim 9, wherein the pairwise comparison is a one class versus all other comparison.

11. The method of claim 1, wherein the supervised learning algorithm is a support vector machine algorithm.

12. The method of claim 11, wherein the support vector machine algorithm is linear or non-linear.

13. The method of claim 1, wherein the steps are performed in a computer system.

14. The method of claim 1, wherein the a digital processor is used to compare the expression pattern of the markers in the sample to the model.

15. In a computer system, a method for classifying at least one biological sample to be tested that is obtained from an individual, wherein expression values of more than one marker are determined for the sample to be tested, comprising:
receiving the gene expression values for more than one marker in the sample to be tested;
providing a model generated by a supervised learning algorithm based on a dataset of expression values from known biological classes;
comparing the gene expression values of the sample to that of the model, to thereby produce a classification of the sample; and
providing an output indication of the classification.

16. A computer apparatus for providing an indication of the classification of a biological sample, wherein the sample is obtained from an individual, wherein the apparatus comprises:
a source of expression values of more than one marker in the sample;
means for providing a model generated by a supervised learning algorithm based on a dataset of expression values from known biological classes;
a processor routine executed by a digital processor, coupled to receive the expression values from the source, the processor routine determining classification of the sample by comparing the expression values of the sample to the model; and
an output assembly, coupled to the digital processor, for providing an indication of the classification of the sample.

17. A method of determining a treatment plan for an individual having a disease, comprising:
obtaining a biological sample from the individual;
providing a model generated by a supervised learning algorithm based on a dataset of expression values from known biological classes;
assessing the sample for the level of expression of more than one marker;
using the model to perform one or more pairwise comparisons of the sample versus at least one disease class, thereby resulting in the disease class classification of the sample; and
using the disease class to determine a treatment plan.

18. A method of determining the efficacy of a drug designed for the treatment of a disease, comprising:
obtaining a biological sample from an individual having the disease;
subjecting the sample to the drug;
assessing the drug-exposed sample for the level of expression of more than one marker;
providing a model generated by a supervised learning algorithm based on a dataset of expression values from known samples on which the drug has different levels of efficacy; and
using a computer to compare the drug-exposed sample to the model to determine the efficacy of the drug in treating the disease.

19. A model produced from a dataset of expression data comprising a plurality of markers from known biological samples formed using a supervised learning algorithm to define a hyperplane that characterizes a biological class.

20. A method of classifying a biological sample comprising:
determining the expression pattern of one or more markers in a biological sample;
providing a model generated by a linear support vector machine algorithm based on a dataset of expression values from multiple known biological classes; and
using a digital processor to compare the expression pattern of the markers in the sample to the model using one or more one versus all other pairwise comparisons,
thereby classifying said biological sample.
